(19) United States
(12) Patent Application Publication
(10) Pub. No.: US 2010/0267124 A1
(43) Pub. Date: Oct. 21, 2010

(54) BIO-ANALYSIS SYSTEM

(76) Inventors: Hisao Inami, Matsudo (JP); Mitsuhiro Matsuzawa, Kyoto (JP); Yasuhiko Sasaki, Tsuchiura (JP)

Correspondence Address: ANTONELLI, TERRY, STOUT & KRAUS, LLP, 1300 NORTH SEVENTEENTH STREET, SUITE 1800, ARLINGTON, VA, 22209-3873, US

(21) Appl. No.: 11/623,780

(22) Filed: Jan. 17, 2007

(30) Foreign Application Priority Data
Feb. 8, 2006 (JP) 2006-030391

(51) Int. Cl. C12M 1/34

(52) U.S. Cl. 435/287.2

(57) ABSTRACT

To improve operability from collection through to gene analysis, and enable the detection of microorganisms in a short time with good accuracy. Provided is an analysis device 400 which conducts germination of microorganism spores and cell membrane lysis with a collecting chip 200 placed in the analysis device 400, and an analysis chip 300 which has a sample reservoir 315, a gene extraction area 320 filled with a gene binding carrier, a waste solution chamber 330 filled with absorbent, washing solution storage chambers 340, 350 which store a washing solution, an eluting solution storage chamber 360 which stores a gene eluting solution, gene-amplification reagent storage chambers 370, 380 which store a gene-amplification reagent, and a reaction chamber 390 which amplifies and detects the gene, each of which is formed by a channel, and a sample injection port 310 and a chip port 241, wherein the treatments from gene extraction to detection are conducted on the analysis chip 300.

### INCORPORATION BY REFERENCE

The present application claims priority from Japanese application JP2006-030391 filed on Feb. 8, 2006, the content of which is hereby incorporated by reference into this application.

### FIELD OF THE INVENTION

The present invention relates to a bio-analysis system which collects microorganisms floating in the air, and extracts a gene contained in the microorganisms to conduct gene analysis of the microorganisms.

### BACKGROUND OF THE INVENTION

To collect microorganisms floating in the air, it is known to suck in air using a portable air-floating germ sampler to collect microorganisms by high-speed impact onto a culture medium provided in a petri dish. This is, for example, described in JP-A-2000-125843.

Further, to provide an inexpensive chip having easy handling, and which enables the process from the extraction of a gene from a sample to the analysis of the gene to be automated in a batch, JP-A-2005-65607 describes the use of a gene-treating chip which comprises an injection port from which the sample containing the gene is fed, a lysis solution section which stores a lysis solution for introducing into the sample fed into the injection port, a gene-extracting section to which a liquid containing the sample and the lysis solution is introduced and which has a gene binding carrier which binds to the gene, a washing solution storage section which stores a washing solution for introducing into the gene-extracting section, an eluting solution storage section which stores an eluting solution for introducing into the gene-extracting section, and a reaction section to which the gene eluted by the eluting section is introduced.

### BRIEF SUMMARY OF THE INVENTION

JP-A-2000-125843 does not describe how the microorganisms collected on a culture medium used for growing the microorganism microstructure and cells are analyzed. Further, even if microorganisms grown on a culture medium are observed to see whether they have multiplied, or employing so-called “detection method by growth”, from 2 to 7 days are required to grow the microorganisms.

Further, in the chip described in JP-A-2005-65607, since the sample containing the gene has to be fed from an injection port, the sample cannot be used as the gene-treating chip from the time of collection, and thus this chip did not allow the operation to conveniently move from collection to gene treating.

Further, since all the gene treatment steps are conducted on a single gene-treating chip, the size of the gene-treating chip increases, which not only leads to a large analyzer, but can also cause costs to increase. In addition, in try to improve analysis accuracy by conducting analysis a plurality of times using the same sample and the same analyzer, all of the treatment steps have to be conducted again, which not only requires a long time, but also means that multiple gene-treating chips will be used, thus causing costs to rise.

It is an object of the present invention to solve the above-described problems of the prior art, by improving the operability from collection through to gene analysis, and enabling the detection of microorganisms in a short time with good accuracy.

It is another object of the present invention to decrease the size and cost of the device while ensuring safety.

Other objects, features and advantages of the invention will become apparent from the following description of the embodiments of the invention taken in conjunction with the accompanying drawings.

### BRIEF DESCRIPTION OF THE DRAWINGS

FIG. 1 is a flowchart illustrating the procedures for bacteria detection as one embodiment of the present invention;

FIG. 2 is a diagram illustrating the structure of a bacteria detection system as one embodiment of the present invention;

FIG. 3 is a diagram illustrating the structure of the main sections of a collecting device;

FIG. 4 is a diagram illustrating the structure of the front end section of a collecting device;

FIG. 5 is a diagram illustrating the planar structure of a collecting chip;

FIG. 6 is a diagram illustrating the cross-sectional structure of a collecting chip;

FIG. 7 is a diagram illustrating the planar structure of an analysis chip;

FIG. 8 is a diagram illustrating the cross-sectional structure of an analysis chip;

FIG. 9 is a diagram illustrating a weir in the gene extraction area of the analysis chip;

FIG. 10 is a diagram illustrating the structure of the waste solution chamber of the analysis chip;

FIG. 11 is a diagram illustrating the structure of the main sections of an analysis device;

FIG. 12 is a diagram illustrating the cross-sectional structure of the analysis device;

FIG. 13 is a diagram illustrating the structure of the substrate of the analysis device; and

FIG. 14 is a diagram showing a profile of liquid handling.

### DESCRIPTION OF REFERENCE NUMERALS

100 Collecting device
110 Lid
120 Nozzle
130 Chip support
150 Support plate
160 Fan motor
170 Exhaust port
180 Controller
181 Display
185 Battery
190 Casing
191 Gripping section
200 Collecting chip
201 Collecting material
241 Chip port
300 Analysis chip
310 Sample injection port
315 Sample reservoir
320 Gene extraction area
330 Waste solution chamber
340 Washing solution A storage chamber
350 Washing solution B storage chamber
360 Eluting solution storage chamber
370 Gene-amplification reagent A storage chamber
380 Gene-amplification reagent B storage chamber
   390 Reaction chamber
   400 Analysis device

### DETAILED DESCRIPTION OF THE INVENTION

To achieve the above-described objects, the present invention provides a bio-analysis system which collects microorganisms floating in air by suction with a collecting device to analyze a gene contained in the microorganisms, comprising: a collecting chip which is placed in the collecting device to collect microorganisms on a collecting material and is then removed from the collecting device and placed in an analysis device; an analysis device which transports a reagent in the collecting chip with the collecting chip placed in the analysis device to conduct in the collecting chip germination of spores in the microorganisms and a cell membrane lysis treatment; and an analysis chip which comprises a sample reservoir, a gene extraction area filled with a gene binding carrier, a waste solution chamber filled with absorbent, a washing solution storage chamber which stores a washing solution, an eluting solution storage chamber which stores a gene eluting solution, a gene-amplification reagent storage chamber which stores a gene-amplification reagent, and a reaction chamber which amplifies and detects the gene, each of which is formed by a channel, and a sample injection port open to a front face and a chip port open to a rear face, wherein a part of the solution treated by the collecting chip is moved to the analysis chip, and the treatments from gene extraction to detection are conducted on the analysis chip by liquid transport means of the analysis device.

Recently, a gene detection method has been adopted which determines whether a microorganism of interest is present or not by amplifying a gene of the microorganism and detecting that gene. For example, bacteria such as Bacillus anthracia or Bacillus cereus form spores within a few hours if they are not surrounded by much water and their nutrients become depleted. These spores are an extremely hard husk-like substance, and are strongly resistant to heat, chemical substances, ultraviolet rays and the like, and thus require a suitable spore treatment. However, the steps of spore-treating or gene extraction from the cell have conventionally been entirely conducted manually. This has not only meant that the operation has been very cumbersome which limits the person performing the operation, but has also involved the risk of that person being infected. In addition, the reaction vessel, pipette and such equipment used in the manual operation all become contaminated waste products, giving rise to the risk of secondary contamination as well as doubts as to the accuracy of the analysis.

An example will now be explained for detecting whether a bacteria of interest is present by collecting bacteria which have formed spores from the air, extracting a gene from the cell after the spores have been treated, and then amplifying the gene by a polymerase chain reaction. The bacteria which form spores include genus Bacillus bacteria, genus Clostridium bacteria and the like.

Flow of Bacteria Detection:

Bacteria detection can be broadly classified into the steps of collecting bacteria, germinating the bacteria spores by adding a germination promoter to the bacteria spores, extracting a gene from the germinated bacteria, and amplifying and detecting the gene. Gene extraction is conducted by a well-known solid-phase extraction method. A “solid-phase extraction method” is a method wherein a gene is specifically bound to a solid surface and then only the gene is eluted into an aqueous solution to differentiate from other substances.

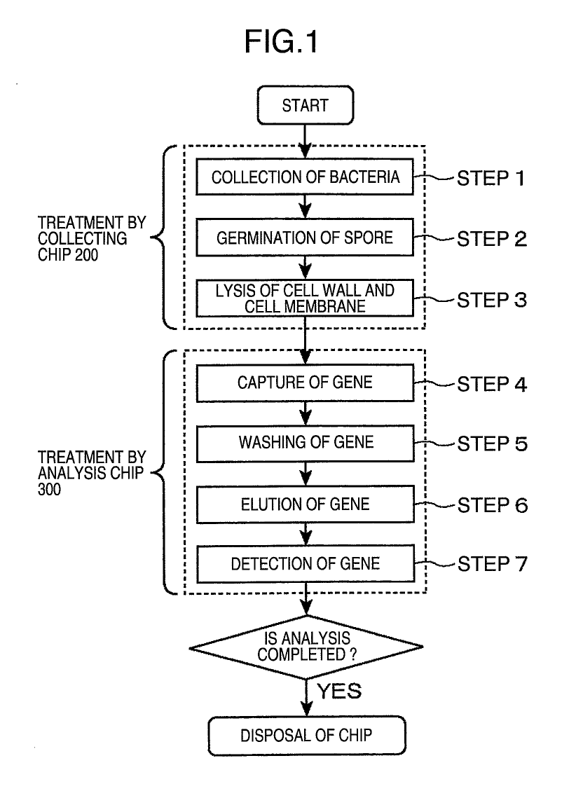

Referring to FIG. 1, a bacteria detection method will now be described.

### Step 1. Collection of Bacteria by Impactor Method:

An “impactor method” collects bacteria on an impactor plate provided below a nozzle by sucking air from above the nozzle and blowing it out the bottom of the nozzle at high speed. The bacteria in the air attain an inertia force in proportion to the square of their particle size, and adhere to the collision plate. This method has the advantages of not causing clogging as in a filter method, and that the bacteria are gathered in a concentrated manner.

### Step 2. Germination of Spore:

A germination promoter is added to the bacteria spores, and after a certain amount of time has passed the bacteria spores start to germinate. At the germination stage, since the bacteria destroy the spores by themselves, the cell walls of the bacteria are in a naked state from germination.

### Step 3. Lysis of Cell Wall and Cell Membrane:

A solution containing chaotropic ions (−1 negative ions having a large molecular diameter) is mixed into the sample, whereby the bacteria cell membranes are disrupted by the action of the chaotropic ions. The chaotropic ions also simultaneously denature a large amount of the proteins contained in the sample, whereby nuclease (an enzyme which breaks down nucleic acids) action is inhibited.

### Step 4. Capture of Gene:

If silica is added into the dissolved mixture, the gene and the silica are specifically bound from the action of the chaotropic ions. Typically, a method is used which passes the mixture through a glass filter.

### Step 5. Washing of Gene:

If proteins or chaotropic ions contained in the sample are mixed into the eluted product, detection of the gene by gene amplification is hindered. Thus, an operation to wash the gene-silica is required. This is normally conducted using highly concentrated ethanol. Since a gene is hardly soluble in such solutions, the gene adsorbed on the silica does not elute in this process.

### Step 6. Elution of Gene:

After the washing, water or a low salt concentration solution is added to the gene-silica to elute the gene from the silica.

### Step 7. Detection of Gene

The eluted gene is charged with a primer (single strand DNAs each having the same base sequence as a sequence of about 20 bases at each end of the intended DNA region), DNA synthetases (polymerases), four types of substrate (dNTP) and the like. By applying the temperature cycle “heat—denaturing—annealing—complementary strand synthesis”, the gene is amplified (polymerase chain reaction). Here, gene amplification can be detected in real time by pre-injecting with a fluorescent dye in addition to the above-described reagent and applying the temperature cycle while irradiating with excitation light.

### Structure of Analysis System

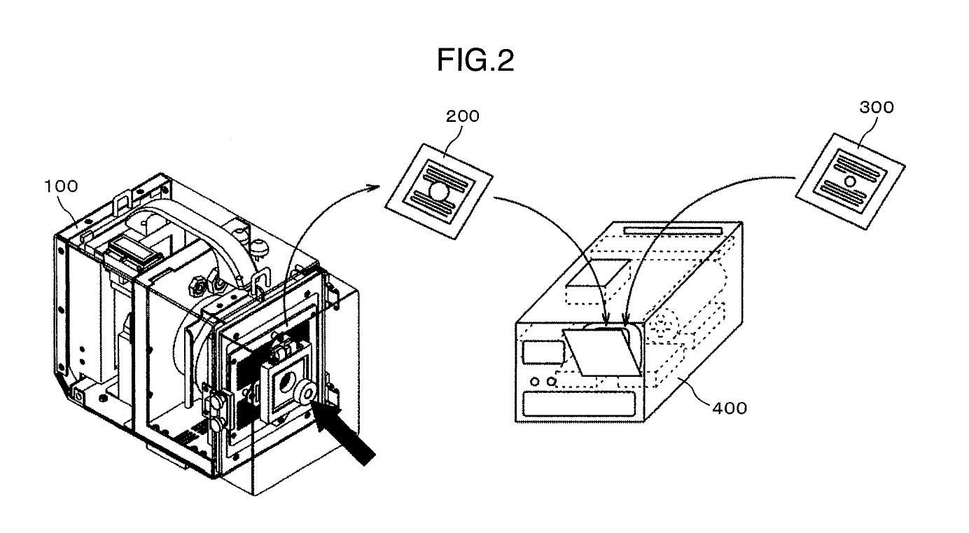

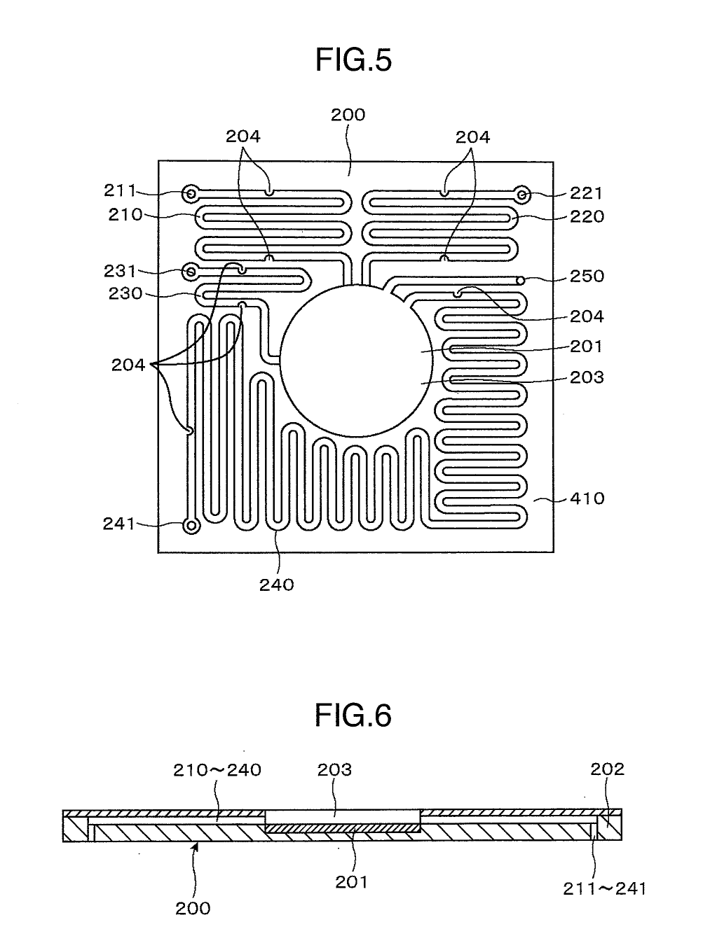

FIG. 2 illustrates the structure of an analysis system, which is comprised from the four components of a collecting device 100, a collecting chip 200, an analysis device 400 and an analysis chip 300. Bacteria floating in the air are sucked in by the collecting device 100 for collection on a collecting material 201 (refer to FIGS. 5 and 6) of the collecting chip 200 mounted in the collecting device 100. The collecting chip 200 is removed from the collecting device 100, and the aperture of the collecting material housing section 203 (refer to FIGS. 5 and 6) of the collecting chip 200 is blocked with a sealant. Alternatively, the collecting chip 200 is removed from the collecting device 100 after the aperture of the collecting chip 200 has been blocked with a sealant, and is then placed in the analysis device 400.

The collecting chip 200 having the above-described aperture sealed by the sealing material is placed in the analysis device 400 with microorganisms adhered to the collecting material. By handling the collecting chip 200 aperture when it is blocked with a sealing material, the collecting chip 200 can be handled safely. The analysis device 400 is equipped with liquid transport means. A reagent in the collecting chip 200 is transported with the collecting chip 200 placed in the analysis device 400. Germination of the bacteria spores and lysis of the cell membranes are performed in the collecting chip 200. Bacteria are collected using the collecting chip 200 which has been pre-embedded with a plurality of reagents. The collecting chip 200 which has collected the bacteria will be used as-is for the next several microorganism detection treatments, which makes it convenient to use from collection to microorganism detection treatment.

The collecting chip 200 is subsequently removed from the analysis device 400, and a part of the liquid processed by the collecting chip 200 is moved to the analysis chip 300 (since the bacteria have already been disrupted by lysis, they cannot cause contamination even if touched).

The analysis chip 300 is placed in the analysis device 400. During this operation, the analysis chip 300 is placed in the same location as the collecting chip 200 had been placed. The analysis chip 300 is pre-embedded with the reagents for Step 4 (Capture of Gene) to Step 7 (Detection of Gene) of FIG. 1. With the analysis chip 300 placed in the analysis device 400, the reagent in the analysis chip 300 is transported by the liquid transport means of the analysis device 400, whereby gene extraction through gene detection is performed in the analysis chip 300. Once the analysis is completed, the analysis chip 300 is removed from the analysis device 400 and discarded.

The reagents necessary for the steps from pre-processing through to detection of the bacteria are all embedded in two kinds of microorganism detection chips (collecting chip 200 and analysis chip 300), whereby cumbersome reagent operations can be obviated. Specifically, in conventional analysis, a reagent is added to the bacteria acting as the sample and then processed, after which the sample is transferred to a separate vessel. As can be seen from this operation, the sample moves through a number of vessels, which is cumbersome and runs the risk of contaminating the testing person. In contrast, the present invention is very safe, since apart from the step of transferring the sample between the two kinds of chip 200 and 300, the sample does not leave the chip, whereby analysis is conducted in a closed system.

Further, the only discarded products are the chips 200 and 300, so that if these chips 200, 300 are made from a material which can be incinerated, the risk of secondary contamination can be reduced. In addition, the reagent embedded in the two kinds of chip 200, 300 are sufficient for only one detection, and thus the chips 200, 300 can be employed as single use chips, which enables high-accuracy bacteria detection at the gene level to be performed simply outdoors.

The two kinds of chip 200, 300 embedded with the reagent are categorized into the collecting chip 200 which conducts processing from collection of the bacteria through to lysis, and the analysis chip 300 which conducts the analytical processing thereafter. This means that it is easy to conduct multiple analyses using a sample having the same processing from collection through to lysis, whereby improvement in accuracy can be easily achieved while maintaining safety.

Structure of Collecting Device:

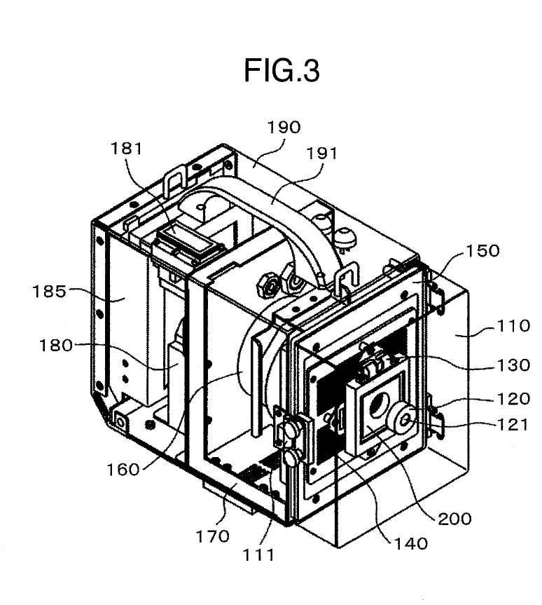

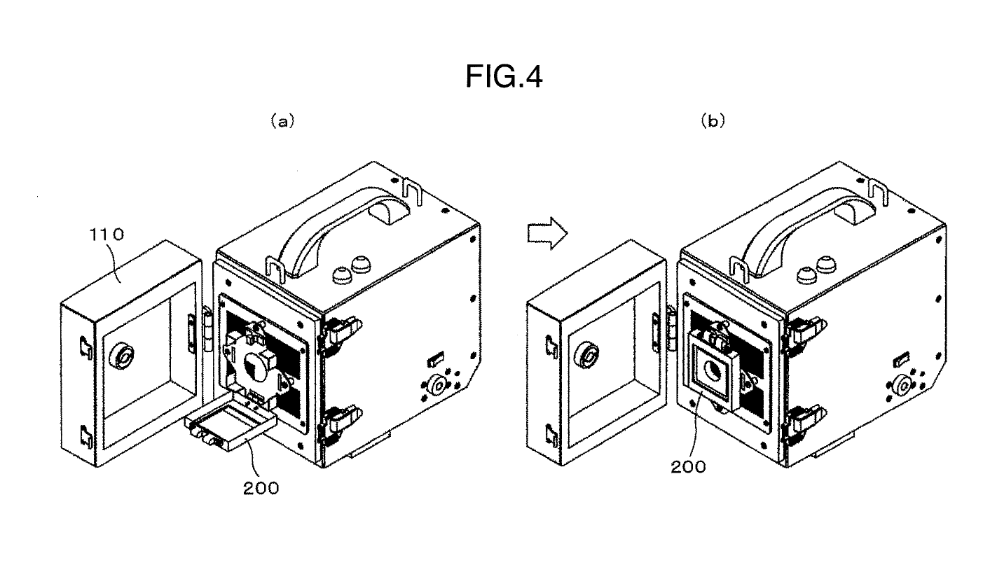

An example of the collecting device according to the present invention will now be explained with reference to FIGS. 3 and 4. FIG. 3 is an oblique perspective view of the collecting device 100 according to the present example. FIG. 4a is a diagram illustrating a chip support 130 with the lid 110 of the collecting device 100 according to the present example open. FIG. 4b is a diagram illustrating a collecting chip 200 mounted with the chip support 130 from FIG. 4a closed.

The collecting device 100 comprises a lid 110, a nozzle 120, a primary filter 121, a chip support 130, a secondary filter 140, a support plate 150, a fan motor 160, an exhaust port 170, a controller 180, a display 181, a battery 185 and a casing 190.

The lid 110 is a square or rectangular section comprising the nozzle 120 and has fixing means on either side. The inner diameter of the nozzle 120 has a strong bearing on collection efficiency. While bacteria can be collected in a more concentrated manner if the nozzle inner diameter is smaller than 10 mm, pressure loss increases as a result of the air flow rate that is flowing through the nozzle 120 increasing. The pressure loss increases in square proportion to the air flow rate, thereby increasing the load on the fan motor 160 and decreasing the current of the battery 185. For example, if the inner diameter of the nozzle 120 is 3 mm or less, the drivable load by the battery 185 (lithium-hydrogen) which can be mounted on the portable bacteria collecting device 100 is exceeded. Therefore, the inner diameter W of the nozzle 120 is preferably 4 to 15 mm, and more preferably 8 to 12 mm. In this range bacteria can be concentrated for collection directly underneath the nozzle 120 while achieving a high collection efficiency.

A primary filter 121 is mounted on the nozzle 120. The primary filter 121 is provided in order to trap coarse particles in the air. Accordingly, its apertures are preferably between 100 and 200 μm. During periods when there is a large amount of pollen in the air, the apertures are preferably between 10 and 100 μm. Within this range, pollen having a particle size of 10 μm or more and bacteria having a particle size of less than 10 μm can be easily separated. The primary filter 121 is detachable from the lid 110, and is preferably made from stainless steel or a fluororesin, which are easily washed and sterilized at high temperatures.

The chip support 130 is mounted on (front) the secondary filter 140. The chip support 130 is retractable (FIG. 4), and if the nozzle 120 is gripped and the lid closed, the collecting chip 200 can be easily placed in the collecting device 100. Since the chip support 130 corresponds to the “periphery” of the collecting chip 200, bacteria are easily adhered. Therefore, the chip support 130 is preferably detachable from the secondary filter 140, and made from stainless steel or a fluororesin, which are easily washed and sterilized at high temperatures.

The secondary filter 140 is mounted on the support plate 150. The secondary filter 140 is provided in order to prevent bacteria fine particles which could not be collected by the collecting chip 200 from being released into the air. The secondary filter 140 preferably employs a HEPA (High Efficiency Particulate Air) filter which can collect at least 99.97% of fine particles having a size of 0.3 μm or more. More preferably, a ULPA (Ultra Low Penetration Air) filter is employed which can collect at least 99.999% of fine particles having a size of 0.1 to 0.2 μm. By using a ULPA filter, the cleanliness of the air released from the exhaust port 170 into the ambient atmosphere can be further increased. The controller 180, display 181 and battery 185 are provided in the casing 190. On an upper face of the casing 190 is provided a gripping section 191.

Next, operation of the collecting device 100 according to the present invention will be described.

If the fan motor 160 is activated, air is sucked into the nozzle 120. The suctioned air is accelerated by the nozzle, and passes through the primary filter 121. Coarse particles in the air are removed at this point by the primary filter 121. The fine particles in the air which has been introduced into the lid 110 are adhered by inertial collision with the collecting material provided in the center of the collecting chip 200. The air introduced into the lid 110 passes through the secondary filter 140, and is externally exhausted from the exhaust port 170 provided on a lower portion of the fan motor 160. Fine particles which were not collected on the collecting chip 200 are removed by the secondary filter 140.

Structure and Operation of Collecting Chip:

An example of the collecting chip 200 will now be described with reference to FIGS. 5 and 6. FIG. 5 is a front view of a collecting chip 200, and FIG. 6 is a cross-sectional view of the collecting chip 200 along its length.

The collecting chip 200 handles from Step 1 (bacteria collection) to Step 3 (cell membrane lysis) of FIG. 1. Specifically, once the collecting chip 200 has been placed in the collecting device 100 and bacteria have been collected (Step 1), the collecting chip 200 is removed from the collecting device 100 and placed in the below-described analysis device 400, and then the processes from Step 2 (spore germination) and Step 3 (lysis of the cell membrane) are conducted.

The collecting chip 200 is prepared with an irregular pattern which models the structural elements of the chip by a photolithography technique. This pattern is a cast which is copied onto the resin by molding. By sticking together two sheets of resin, the patterns carved into the resin form a channel. As the chip material, it is preferable to use a resin, which has excellent disposability, rather than glass which is expensive to work and is easily broken. The kind of resin is not especially limited, but the use of polydimethylsiloxane (PDMS: manufactured by Dow Corning Asia, Silpot 184) is preferable, so that the chip preferably comprises the following characteristics:

Excellent biocompatibility (ordinary silicone rubber is physiologically inert)

Copying of pattern can be effected with submicron precision (before curing, it has low viscosity and high fluidity, and thus can favorably permeate into the intricacies of complicated shapes)

Low cost (8 yen/gram. Less than a one-hundredth of that of conventional general-purpose material, Pyrex glass, for microdevices which is 1000 yen/gram)

Easily disposable by incineration

The collecting chip 200 comprises the collecting material 201 on which microorganisms (in the present embodiment, bacteria which have formed spores) from the air have adhered for collection, and a thin plate-shaped substrate mounting the collecting material 201. The collecting chip 200 has a collecting material housing section for housing the collecting material 201, a plurality of reagent storage chambers (210, 220, 230, 240), chip ports 211, 221, 231, 241 which are open to the chip rear face, and an air opening 250 which is open to the chip front face.

The reagent storage chambers comprise a germination promoter storage chamber 210 which stores a germination promoter, an enzyme A storage chamber 220 which stores two kinds of cell wall lysis solution, an enzyme B storage chamber 230, and a chaotropic storage chamber 240 which stores chaotropic ions. The plurality of reagent chambers 210 to 240 are provided so as to encompass the periphery of the collecting material housing section, thereby enabling the collecting chip 200 to be made compact.

The reagent chambers 210 to 240 are configured from long, thin channels, and it is preferable for all of the reagent chambers to be in a shape having a channel. To transport a reagent to the reagent chambers 210 to 240, gas is sent from the rear side of the reagent chambers 210 to 240 to the reagent chambers 210 to 240. If the reagent chambers 210 to 240 are formed in a long, thin channel shape, a reagent is only pushed out of locations where the gas can easily pass through, meaning that a reagent remains in the reagent chamber at the other locations. To lower the amount of a reagent being consumed, it is effective for the reagent chambers 210 to 240 have a channel shape.

The cross-sectional shape of a channel preferably has a width to length ratio of no greater than 10. If the width to length ratio is greater than 10, the resin of the channel ceiling may sag thereby destroying the rectangular shape of the channel, which can become an impediment to transportation. The long, thin channel of the reagent chambers 210 to 240 is formed in a serpentine shape to ensure storage capacity of the reagent in the channel while reducing the area that the channels occupy on the substrate.

One end of the reagent chambers 210 to 240 is connected to the collecting material housing section 203, and the other end of the reagent chambers 210 to 240 is connected in communication with a chip port (211 to 241). The one end of the reagent chambers 210 to 240 and the other end of the reagent chambers 210 to 240 are each provided with a weir 204, whereby the flow of the reagent stored in each of the reagent chambers 210 to 240 can be reliably stopped.

The chip ports 211 to 241 form junctions with the external channels. The germination promoter storage chamber 210, enzyme A storage chamber 220, enzyme B storage chamber 230 and chaotropic storage chamber 240 are all in communication with the collecting material housing section 203. Therefore, the collecting material housing section 203 is connected to external channels via the germination promoter storage chamber 210, enzyme A storage chamber 220, enzyme B storage chamber 230 and chaotropic storage chamber 240 and the chip ports 211 to 241. By narrowing the channel width of the reagent chambers 210 to 240 to 50 to 100 μm, the influx of air can be prevented from the collecting material housing section 203 side.

The volume of the germination promoter storage chamber 210 is preferably 20 to 100 μL, the volume of the enzyme A storage chamber 220 is preferably 20 to 100 μL, the volume of the enzyme B storage chamber 230 is preferably 5 to 20 μL, and the volume of the chaotropic storage chamber 240 is preferably 400 to 800 μL. Disruption of the cell membranes is promoted by making the volume of the chaotropic ions at least twice the total volume of the two kinds of germination promoter. More preferred is 4 times or greater, and optimally, 8 times or greater.

Agar having free water on the gel surface (water among the gel network) is preferred as the collecting material 201. The agar concentration is preferably 2 to 5%, and most preferably 3 to 4%. If the agar concentration is less than 2%, the moisture content is large, meaning that its strength as the collecting material 201, on which high-speed air is continuously applied, is insufficient. On the other hand, if the agar concentration is greater than 6%, the moisture content (free water) of the agar surface is reduced, whereby adhesion dramatically decreases.

To increase the strength of the agar and stop the moisture from evaporating, it is effective to add an alcohol, which acts as an antifreeze agent, drying-prevention agent and a gel strengthening agent. Specific examples thereof include ethyl alcohol, isopropyl alcohol, 1,3-butandiaol, ethylene glycol, propylene glycol, glycerin and the like. The added amount of alcohol is preferably 40 to 80% of the agar, and more preferably 50 to 70%. If the added amount is less than 40%, evaporation prevention of moisture is insufficient, and if the added amount is more than 80%, the moisture content (free water) of the agar surface is reduced, whereby adhesion decreases.

One example of a method for using the collecting chip 200 will now be described.

The collecting chip 200 is attached to the chip support of the collecting device 100 so that air is sucked in for a certain period of time. The amount of suctioned air is, for example, about 1,000 L. Bacteria in the air adhere to the collecting material 201 surface of the collecting chip 200. Next, the collecting chip 200 is taken off the chip support. After the aperture surface of the collecting material housing section 203 of the collecting chip 200 has been sealed, the collecting chip 200 is placed in the analysis device 400. While sealing of the collecting material housing section 203 may be conducted manually, is preferred to have a sealing mechanism on the collecting device. Sealing the collecting material housing section 203 prevents bacteria being externally exposed from the collecting chip 200, thereby improving safety.

The reagent chambers 210 to 240 of the collecting chip 200 are connected to external channels via chip ports (211 to 241). Thus, by feeding gas via the chip ports 211 to 241 by a predetermined control operation from the channel side of the analysis device 400, the germination promoter, cell wall lysis solution, cell membrane lysis solution and chaotropic ions encapsulated in the reagent chambers 210 to 240 are transported at predetermined times into the collecting material housing section 203 (onto the collecting material 201). Although the upper front face of the collecting material housing section 203 is sealed, because an air opening 250 is in communication with a part of the collecting material housing section 203 and thus open to the air, when transporting the reagent air present above the collecting material 201 is released from the air opening 250.

Details of the transportation of the reagent from the reagent chambers 210 to 240 to the collecting material 201 will now be described.

100 μL of the germination promoter is transported to the collecting material 201. Here, the germination promoter is preferably a broth comprising alanine, adenosine or glucose. A broth comprising 1 mM to 10 mM of L-alanine is especially preferable. After 10 minutes have passed, germination of the bacteria spores starts, and after 30 minutes have passed, at least 50% have germinated. Therefore, the spore germination treatment is preferably no less than 30 minutes. A preferable temperature to cause the bacteria spores to germinate is 35 to 40° C., and most preferred is 35 to 37° C. Since the bacteria destroy the spores by themselves during the stage wherein the bacteria spores germinate, the cell walls of the bacteria are in a naked state from germination.

Next, the two kinds of protein-modifying enzyme are successively transported to the collecting material 201, and an optimum temperature is maintained for a certain period of time. The duration of the enzyme treatments is preferably respectively 10 minutes or more, and 30 minutes is suitable. Here, as the protein-modifying enzyme, 100 μL of a lysozyme (an optimum temperature of 37° C.) and 20 μL of protease K (an optimum temperature of 55 to 60° C.) is suitable. As a result of these enzyme treatments, the bacteria in the collecting chip 200 have their cell membrane bared. Further, while the germination promoter and the lysozyme can be introduced at the same time, the lysozyme and the protease K should not be added at the same time, as this reduces enzyme activity.

Finally, by transporting 800 μL of chaotropic ions to the collecting material 201, the bacteria cell membranes are disrupted, whereby a bacteria gene is released out of the cells. Examples of chaotropic ions include guanidine thiocyanate, guanidine hydrochlorate, sodium iodide, potassium bromide and the like. Examples of the method for using the chip include maintaining the activity of the reagents for a long period of time by refrigerating or freezing the chip. Therefore, guanidine hydrochlorate is preferable, as it shows very little change in structure when refrigerated or frozen.

It is also preferable to incorporate a surfactant or a buffering agent in the chaotropic salt. The surfactant is not especially limited, and may be Twin-20, Triton X-200 and the like. The buffering agent is not especially limited, and may be tris-hydochloride, potassium dihydrogen phosphate, sodium borate and the like.

According to the above steps, germination of the cells collected on the collecting chip 200 and treatment of the cell walls can be conducted. Specifically, as the operation can be automated on the chip as far as Steps 2 or 3 illustrated in FIG. 1, it is no longer necessary to dispense the reagents separately. The collecting chip 200 handles the steps from the collection of bacteria to pretreatment, and the analysis chip 300 handles the step of analyzing the bacteria gene. To increase the accuracy of the analysis, it is preferable to analyze the same sample multiple times. Alternatively, to set a plurality of target bacteria, it is preferable to divide a sample treated by a single collecting chip 200 among plural analysis chips 300, so that two kinds of chip 200, 300 are provided.

It is noted that if the collecting chip 200 is given to a user while frozen and the user stores the analysis chip 300 frozen at 0° C., the activity of the reagent can be maintained for one month. In addition, by storing frozen at −20° C., the activity of the reagent can be maintained for half a year or longer.

Structure and Operation of Analysis Chip:

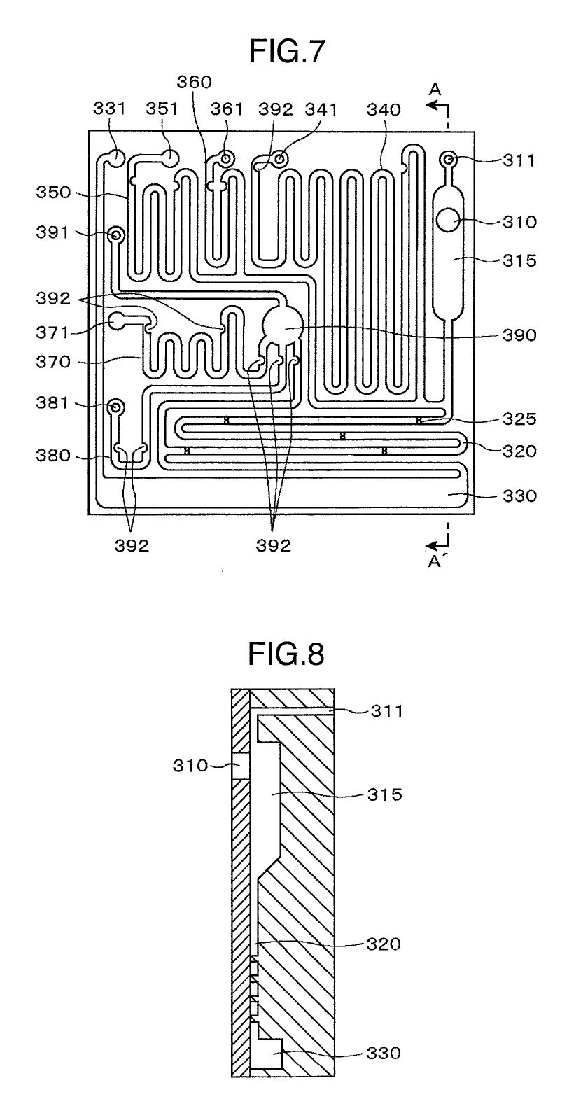

The analysis chip 300 will now be specifically described with reference to FIGS. 7 to 10. FIG. 7 is a front view of the analysis chip 300, and FIG. 8 is a cross-sectional view taken along the line A-A′ of FIG. 7. FIG. 8 is a cross-sectional view of when the analysis chip 300 is placed upright.

The analysis chip 300 handles from Step 4 (Capture of Gene) to Step 7 (Detection of Gene) of FIG. 1. A part of the liquid treated by the collecting chip is placed in an analysis device 400 which has been moved. The reagents which will be used in Step 4 (Capture of Gene) to Step 7 (Detection of Gene) of FIG. 1 are pre-embedded in the analysis chip 300. With the analysis chip 300 placed in the analysis device 400, the liquid transport means on the analysis device 400 is activated to transport the reagents in the analysis chip 300, and the treatments from gene extraction to gene detection are conducted in the analysis chip 300. The material for the analysis chip 300 is the same resin as that of the collecting chip 200.

The analysis chip 300 comprises a sample injection port 310 open to the chip front face, a sample reservoir 315, a gene extraction area 320 filled with a gene binding carrier in the channel, a waste solution chamber 330, a washing solution A storage chamber 340 which stores a washing solution A, a washing solution B storage chamber 350 which stores a washing solution B, an eluting solution storage chamber 360 which stores a gene eluting solution, a gene-amplification reagent A storage chamber 370 which stores a gene-amplification reagent A, a gene-amplification reagent B storage chamber 380 which stores a gene-amplification reagent B, a reaction chamber 390 which amplifies and detects the gene, and chip ports 311, 331, 341, 351, 361, 371 and 381 which are open to the chip rear face. Similar to the collecting chip, the cross-sectional shape of these reagent chambers 340 to 380 preferably has a width to length ratio of no greater than 10, and are formed with a width of 2 mm and a length of 3 mm. On the other hand, the cross-section of the channels other than the reagent chambers 340 to 380 is preferably smaller than that of the reagent chambers 340 to 380, and a cross-sectional area of ¼ is preferable. The analysis chip 300 is fabricated by copying from a resin mold fabricated by a stereolithography. Since it is difficult to form a resin fabricated by a stereolithography with smooth curves, a rectangular structure is generally used. Thus, the channel cross-section of an analysis chip copied from a resin mold having a rectangular structure will naturally be rectangular. When a reagent is flowed through a rectangular channel, the reagent tends to adhere to the four corners of the channel and remain there. Since the reagent remaining in the channel mixes with the reagent which is flowed through next, analysis accuracy deteriorates. Accordingly, because reagent adherence to the channel wall surfaces can be suppressed and “carry over” of the reagent prevented by decreasing the cross-section of the channel, the width is 0.5 mm and the length is 0.5 mm. Further, so that the reagent in the reagent chambers 340 to 380 does not flow out by itself when the analysis chip is placed upright, the reagent storage chambers may be formed in a “U” shape or the channels in communication with the reagent storage chambers may be once made to face upwards.

The chip ports 311 to 381 are formed on one end of the sample reservoir 315, waste solution chamber 330 and reagent chambers 340 to 380. The chip ports 311 to 381 act as junctions with the channels of the external analysis device 400. To transport the reagent in the reagent chambers 340 to 380, gas from the analysis device 400 which is external to the chip is sent to the reagent chambers 340 to 380 via the chip ports 341 to 381. Since oxygen can oxidize the reagent, and carbon dioxide can change the pH of the reagent, preferable examples of the gas are inert, such as nitrogen, helium, argon or the like.

The reagent reservoir 315, washing solution A storage chamber 340, washing solution B storage chamber 350 and eluting solution storage chamber 360 are all in communication with the gene extraction area 320. Mixing of the sample or washing solution A with the lysis solution inhibits the detection of the gene, so that it is preferable to position the eluting solution storage chamber 360 away from the sample reservoir 315 and the washing solution A storage chamber 340.

The gene-amplification reagent A storage chamber and the gene-amplification reagent B storage chamber are in communication with the reaction chamber 390. Once the gene-amplification reagent A sent to the reaction chamber 390 from the gene-amplification reagent A storage chamber 370 and the gene-amplification reagent B sent to the reactor from the gene-amplification reagent B storage chamber have been introduced into the reaction chamber 390, the gene-amplification reagent A storage chamber and the gene-amplification reagent B storage chamber are in communication through the top of the reaction chamber 390 to prevent reflux from the reactor.

The sample reservoir 315 preferably has a volume of 100 to 200 μL, the gene extraction area 320 preferably has a volume of 100 to 200 μL, the washing solution A storage chamber 340 preferably has a volume of 200 to 300 μL, the washing solution B storage chamber 350 preferably has a volume of 50 to 100 μL, the eluting solution storage chamber 360 preferably has a volume of 10 to 20 μL, the gene-amplification reagent A storage chamber 370 preferably has a volume of 20 to 40 μL and the gene-amplification reagent B storage chamber 380 preferably has a volume of 10 to 20 μL.

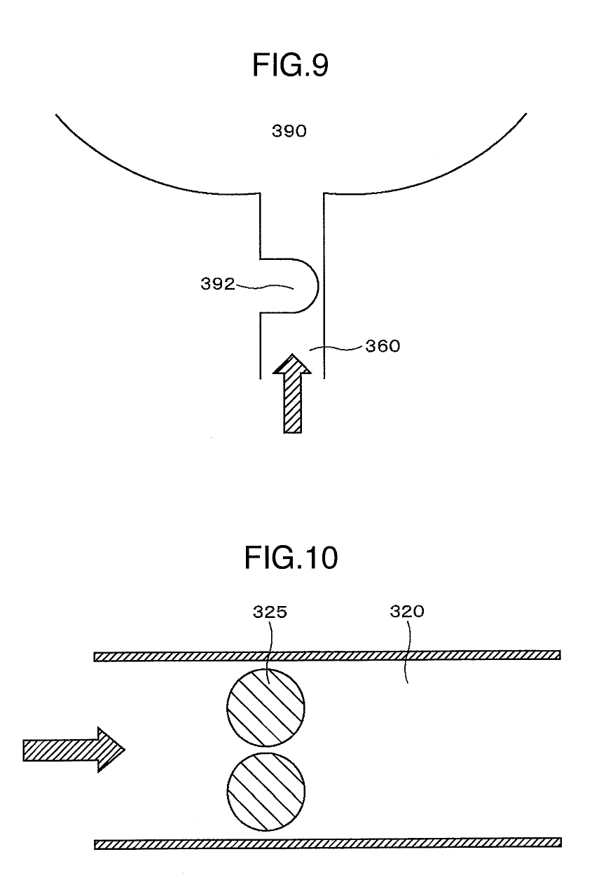

Examples of materials which may be employed as the gene binding carrier filled into the gene extraction area 320 include quartz wool, glass wool, glass fiber and glass beads. If using glass beads, to increase contact surface area the size of the beads is preferably no greater than 50 μm, and in consideration of the blockages in the channel, 20 to 30 μm is most preferable. To block the gene binding carrier, it is preferable to arrange a weir 325 in at least one location in the channel which forms the gene extraction area 320. FIG. 9 illustrates one example of the structure of the weir. In the gene extraction area 320 channel, by narrowing the channel width to 15 to 20 μm at several locations, the narrowed channel acts as a weir against the gene binding carrier. If the channel is made narrower than 10 μm, flow resistance is large, whereby flow control becomes difficult. Therefore, the channel width as the weir 325 is preferably 15 to 50 μm.

The solutions which flow to the waste solution chamber 330 as waste solutions are the sample, the washing solution A and the washing solution B. While these solutions do not directly effect the human body, it is necessary to make the structure such that such solutions cannot escape out of the waste solution chamber 330. FIG. 10 illustrates one example of the structure. The example illustrates that two kinds of absorbent 332, 333 are filled in the waste solution chamber 330. Preferable materials as the absorbent 332 should have little volume expansion, such as low-cost cotton, washi (Japanese paper) or the like. As the second absorbent 333, acrylamide is most preferred. Although acrylamide is somewhat more expensive than cotton or Japanese paper, it has very high absorption capacity and its volume dramatically expands in conjunction with the absorbing solution. Specifically, the acrylamide volume expands while absorbing the waste solutions in the waste solution chamber, which restricts the volume of the waste solution chamber, whereby the pressure in the waste solution chamber rises. As is described below, the analysis chip has a section which bifurcates the reagent immediately prior to the waste solution chamber. To ensure that a reagent does not enter the waste solution chamber, it is necessary to raise the pressure above that of the bifurcating-side channel. Namely, the reason for filling the waste solution chamber with absorbent is not only to absorb so that waste solution does not leak from the analysis chip, but also to perform the role of actively adjusting the pressure inside the waste solution chamber by volume expansion of the adsorbent. For that reason, it is preferable to employ a smaller amount of acrylamide as the absorbent 333 than the amount of absorbent 332.

In addition, as the means for suppressing waste solution leakage from the waste solution chamber 330, it is effective to subject the interior of the waste solution chamber 330 to a hydrophilic treatment. Since the resin material used as the chip material is originally hydrophobic, waste solution which has entered the waste solution chamber 330 is repelled by the wall surfaces inside the waste solution chamber 330, thereby rendering it difficult to completely fill the waste solution chamber 330 with waste solution. Thus, by subjecting the interior of the waste solution chamber 330 to a hydrophilic treatment, the wettability of the solution are good, and the waste solution can be filled from the bottom of the waste solution chamber 330. When fabricating the analysis chip, the resin surface can be modified by irradiating plasma onto the waste solution chamber 330, whereby it is easy to make the waste solution chamber 330 hydrophilic.

Structure and Operation of Analysis Device:

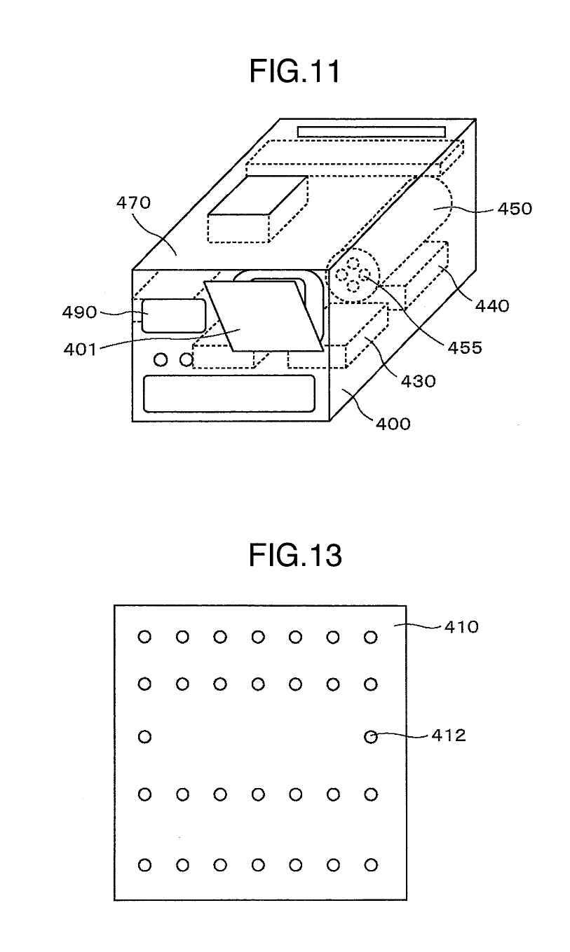

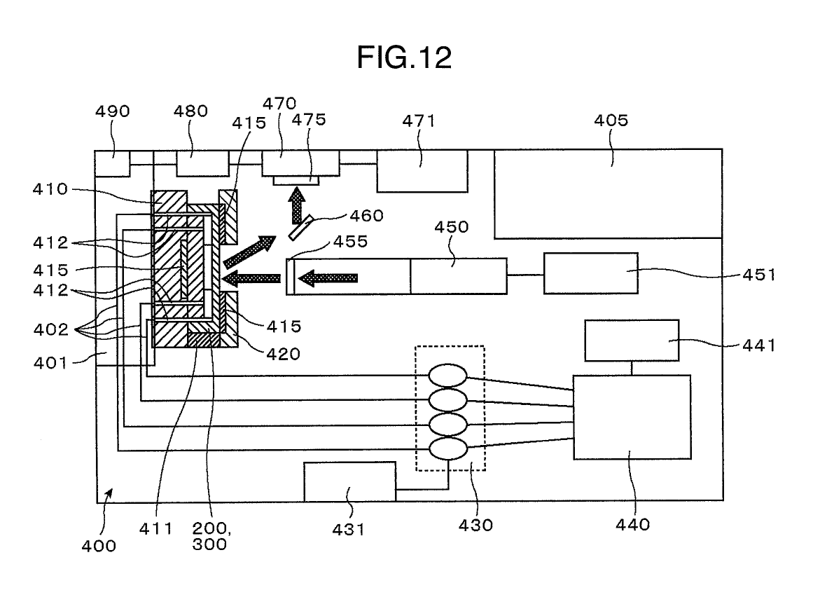

The structure and operation of the analysis device 400 will now be described with reference to FIGS. 11 to 13. FIG. 11 is a diagram illustrating the main structure of the analysis device 400; FIG. 12 is a diagram illustrating the cross-sectional structure of the analysis device 400; and FIG. 13 is a diagram illustrating the substrate 410 of the analysis device 400.

The analysis device 400 is roughly divided into four systems: a chip positioning section, a fluid system, a temperature control system, and an optical detection system. The substrate 410 on which the collecting chip 200 and analysis chip 300 are placed is provided on an inner side of the fore lid 401. Since both chips are placed in an upright manner, a chip stopper 411 for stopping the chips is provided at a lower portion of the substrate 410. If the chips are placed on the substrate 410 and the fore lid 401 is closed, the chips are fixed in between the chip substrate 410 and the chip holder 420. The chip substrate 410 and the chip holder 420 are provided with a temperature control mechanism 415 for optimizing the temperature of the chips. As the temperature control mechanism 415, a Peltier device is suitable as the heat generator, whereby the heating/cooling operation can be easily conducted just by changing the direction of the applied current.

The substrate 410 is provided with a substrate channel 412. One end of the substrate channel 412 is in communication with a chip port, while the other end of the substrate channel 412 is in communication with a device internal channel 402. The substrate is provided in advance with a plurality of substrate channels 412, so that the substrate channel 412 can be applied to the chip ports of either the collecting chip 200 or the analysis chip 300, whereby the analysis device 400 can become a platform for the collecting chip 200 and the analysis chip 300.

The device internal channels 402 are respectively connected to a pump 440 via a valve 430. To transport the reagent in the reagent chamber in a given chip, the valve 430 is switched to blow only along the channel in communication with that reagent chamber. The gas sent by the pump 440 is delivered into the chip via the selected device internal channel 402 and substrate 410 channel, whereby the reagent in the reagent chamber is transported. Since a predetermined amount of a reagent is pre-embedded in the reagent chamber, the reagent in the reagent chamber may be just discharged by time management, thus obviating the need for transport accuracy of the pump 440. Accordingly, the pump 440 only blows and does not suck, so that a simple and compact device may be used.

The valve 430 that controls the fluid is preferably provided at the analysis device 400 side rather than inside the chip. By so doing, the chip 101 becomes free of mechanical parts, thereby attaining size-reduction and disposability.

The optical detection system is composed of a light source 450 that irradiates an excitation light onto the gene in the chip reaction chamber 390, an excitation filter 455 which only allows through excitation light having a specific wavelength, a mirror 460 which changes the light path of the fluorescence generated from the chip reaction chamber 390, a detection filter 475 which only allows through fluorescence having a specific wavelength, and a photodetector 470 which measures fluorescence. As the light source 450, while devices having different wavelength regions may be used, a xenon lamp is used which has a broad wavelength region. In cases where the wavelength is limited, an LED may be used. Examples of the photodetector 470 include a CCD camera, a photomultiplier tube, a photodiode and the like, with the photodiode being preferred in order to reduce the size of the device. Light signals of the gene detected by the photodetector 470 are digitized by a light signal converter 480, and the signal intensity is displayed on the data display screen 490.

The analysis device 400 is provided with control mechanisms for performing each control. On the analysis device 400 are mounted a valve control mechanism 431 which controls the valve 430, a pump control mechanism 441 which controls the pump 440, a light source control mechanism 451 which controls the light source 450, and a photodetector control mechanism 471 which controls the photodetector 470. Thus, a compact and portable analysis device can be provided by placing a compact analysis chip which is free from mechanical parts on the substrate, and combining with a simple photodetector.

Analysis Procedure:

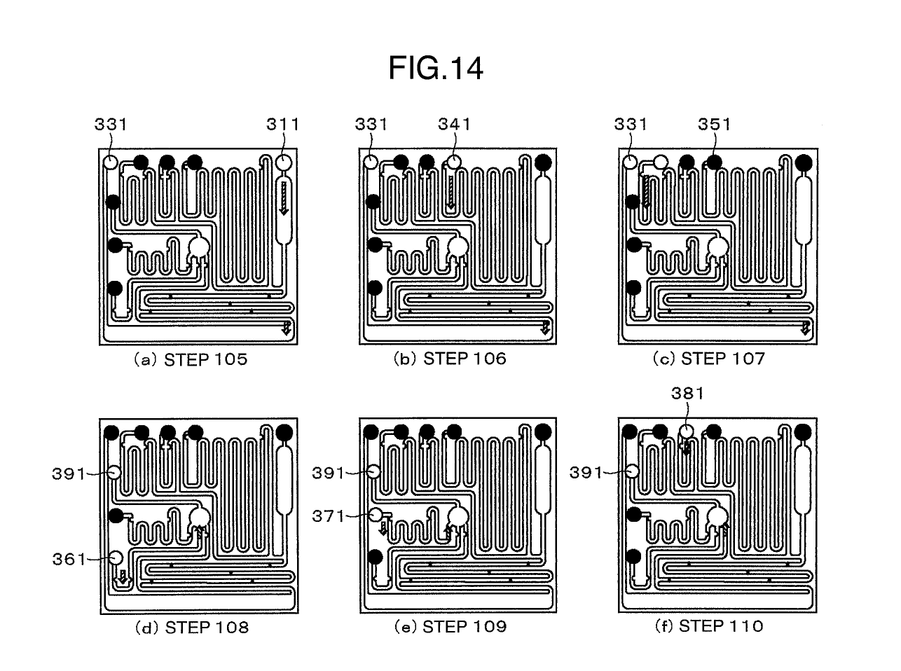

The analysis procedure using the analysis chip 300 and the analysis device 400 will now be described with reference to FIG. 7, FIG. 12, and FIG. 14. FIG. 14 is a diagram showing a profile of fluid handling.

The analysis procedure using the analysis chip 300 mainly has the following procedures.

First, a bacteria sample whose cell walls have been dissolved by the collecting chip 200 is injected into the analysis chip 300. At the analysis chip 300, the bacteria sample is transported to a channel filled with a gene-retaining carrier. Then, a washing solution which washes proteins etc. contained in the sample is transported to the channel filled with the gene-retaining carrier.

Next, an eluting solution which elutes the gene adsorbed to the gene-retaining carrier is transported to the channel filled with the gene-retaining carrier, and further transported to a reaction chamber in which the gene is detected.

Then, the presence of the gene of a target for analysis is detected. An example is specifically described below.

First, an analysis chip, that had been refrigerated or frozen, and which has built-in a washing solution A storage chamber 340, a washing solution B storage chamber 350, an eluting solution storage chamber 360, a gene-amplification reagent A storage chamber 370, and a gene-amplification reagent B storage chamber 380, which respectively contain five kinds of reagents, i.e. a washing solution A, a washing solution B, a gene eluting solution, a gene-amplification reagent A and a gene-amplification reagent B, is thawed at room temperature. By providing a user with a pre-embedded reagent for only one detection in the analysis chip 300, the analysis chip 300 can be rendered as a single-use chip which does not waste any reagent, thereby improving cost effectiveness. Also, the user can obviate the work required to deliver the reagents into each reagent storage chamber, which can not only shorten time, but can prevent contamination. Furthermore, by providing the user with the analysis chip 300 in a frozen state, if the user stores the analysis chip 300 frozen at 0° C., the activity of the reagent can be maintained for one month. Also, by storing frozen at −20° C., the activity of the reagent can be maintained for half a year or longer. Thus, by providing the user with a disposable analysis chip 300 pre-embedded with a reagent for one detection in a refrigerated or frozen state, a simple analysis environment can be created (Step 101).

After the analysis chip 300 has been thawed, approximately 100 μL of the collecting chip 200 treating solution is sent to the sample injection port 310 of the analysis chip 300 (Step 102).

A cover is then put over the sample injection port 310 to block the aperture. As the cover, a thin resin sheet of the same material as the analysis chip 300 is preferable. Since adhesion between the resins is good, and the resin is low cost, this is suitable for a single-use chip. While the step of covering the sample injection port 310 may be conducted manually, it is more preferable to have on the analysis device 400 side a mechanism which covers the sample injection port 310 (Step 103)

Next the fore lid 401 of the analysis device 400 is opened, and the analysis chip 300 is set into the analysis device 400 by following the chip guide provided on the fore lid 401, and the fore lid 401 of the analysis device 400 is then closed. By doing this, the analysis chip 300 is fixed onto the substrate 410, and the chip port and the device internal channel 402 are in communication with each other. While the analysis chip 300 may be placed either on its side or in an upright manner, the example described here is explained for when it is in an upright manner (Step 104).

Subsequently, by switching the valve 430 in the analysis device 400, a fluid is run from the pump 440 only to the sample port 311 (ports 311, 331: open; other ports: closed). The fluid used here may be any kind of fluid such as air or nitrogen as long as it does not deteriorate the activity of the reagent when contacted with the reagent. The reagent in the sample reservoir 315 moves to the gene extraction area 320. As a result of the action of the chaotropic ions in the sample, the bacteria gene in the sample bind to the gene binding carrier filling the gene extraction area 320. To promote the binding between the bacteria gene and the gene binding carrier, the sample is preferably passed through the gene extraction area 320 for 10 minutes or more. The sample which has passed through the gene extraction area 320 accumulates in the waste solution chamber 330. The gas used for transportation is discharged from the waste solution port 331. It is noted that by placing the chip in an upright manner, the sample can be prevented from leaking from the waste solution port 331 (Step 105).

Subsequently, by switching the valve 430 in the analysis device 400, closing the port 311 and opening the washing solution A port 341, a fluid is run from the pump 440 only to the washing solution A port 341 (ports 331, 341: open; other ports: closed). 200 μL of the washing solution A in the washing solution A storage chamber 340 is transported to the gene extraction area 320 by the fluid. Preferred examples of the washing solution A include chaotropic ions such as guanidine thiocyanate, guanidine hydrochlorate, sodium iodide, potassium bromide or the like. Proteins remaining in the gene extraction area 320 as a result of the washing solution A are removed. The washing solution A which has passed through the gene extraction area 320 accumulates in the waste solution chamber 330 (Step 106).

Subsequently, by switching the valve 430 in the analysis device 400, closing the port 341 and opening the washing solution B port 351, a fluid is run from the pump 440 only to the washing solution B port 351 (ports 331, 351: open; other ports: closed). 50 μL of the washing solution B in the washing solution B storage chamber 350 is transported to the gene extraction area 320 by the fluid. Preferred examples of the washing solution B include highly concentrated ethanol of 50% or greater, potassium acetate or the like. Chaotropic ions remaining in the gene extraction area 320 as a result of the washing solution B are removed. The washing solution B which has passed through the gene extraction area 320 accumulates in the waste solution chamber 330 (Step 107).

Subsequently, by switching the valve 430 in the analysis device 400, closing the ports 331 and 351, and opening the eluting solution port 361 and the reaction chamber port 391, a fluid is run from the pump 440 only to the eluting solution port 361 (ports 361, 391: open; other ports: closed). 10 μL of the eluting solution in the eluting solution storage chamber 360 is transported to the gene extraction area 320 by the fluid. As the eluting solution, sterilized distilled water, a buffer solution such as TRIS-EDTA and TRIS-acetate or the like can be used. The gene trapped by this eluting solution on the gene binding carrier in the gene extraction area 320 are eluted. The eluted gene is transported to the reaction chamber 390.

Subsequently, by switching the valve 430 in the analysis device 400, closing the port 361, and opening the gene-amplification reagent A port 371, a fluid is run from the pump 440 only to the gene-amplification reagent A port 371 (ports 371, 391: open; other ports: closed). 10 μL of the gene-amplification reagent A in the gene-amplification reagent A storage chamber 370 is transported to the reaction chamber 390 by the fluid.

The gene-amplification reagent A is composed of four types of dNTP (dATP, dCTP, dGTP, dTTP), a buffer (Tris-HCl, KCl, MgCl₂), and a primer. The gas used for transportation is discharged from the reaction chamber port 391 (Step 108).

Subsequently, by switching the valve 430 in the analysis device 400, closing the port 371, and opening the gene-amplification reagent B port 381, a fluid is run from the pump 440 only to the gene-amplification reagent B port 381 (ports 381, 391: open; other ports: closed). 30 μL of the gene-amplification reagent B in the gene-amplification reagent B storage chamber 380 is transported to the reaction chamber 390 by the fluid. The gene-amplification reagent B is composed of DNA synthetases (such as Taq DNA polymerase, Tth DNA polymerase, Vent DNA polymerase, and thermosequenase), and a fluorescent dye (such as ethidium bromide, and SYBR GREEN (manufactured by Molecular Probe) FAM and ROX) (Step 109).

Next, Step 110 is conducted.

In accordance with the above procedures, the bacteria gene and two kinds of gene-amplification reagent have been introduced into the reaction chamber 390 of the analysis chip 300.

Then, to amplify and detect the bacteria gene in the reaction chamber 390, the temperature control mechanism 415 is activated, and a temperature cycle is applied so that the temperature of the reaction chamber 390 moves back and forth between the following two predetermined values (Step 111).

As an example of temperature cycle, roughly the following may be performed:

[90 to 95° C. for 10 to 30 seconds←→65 to 70° C. for 10 to 30 seconds]×30 to 45 times

As a preferred example, the following temperature cycle may be performed:

[94° C. for 30 seconds←→68° C. for 30 seconds]×45 times

While performing the temperature cycle, an excitation light is irradiated onto the reaction chamber 390 from the light source 450. The gene, if it has a fluorescent dye intercalated into the inside of the double strand, transfers the energy of the absorbed light of the light source 450 to the fluorescent dye (energy transfer). As a result, the fluorescent dye is excited and emits fluorescence. Thus, when the gene of interest is present in the sample, the amount of fluorescence emitted increases as the gene is amplified. Therefore, by monitoring the amount of fluorescence in the reaction chamber 390 by the photodetector 475 during the temperature cycle, the presence or absence of the gene of interest can be detected in real time as shown in FIG. 7. Placing the analysis chip 300 in the analysis device 400 in an upright manner has the advantage of preventing a decrease in the detection sensitivity, because the reaction chamber 390 side face which detects the fluorescence does not cloud up even if a part of the reaction substance evaporates during the temperature cycle to cause vapor to accumulate at an upper section of the reaction chamber 390 (Step 112).

After analysis is completed, the analysis chip 300 is removed from the analysis device 400, and discarded (Step 113). This obviates the need for post-treatment of the samples and the reagents and the need for a washing procedure of the reaction detection section, thereby enabling simple and rapid analysis to be provided.

By using a collecting chip and analysis chip in combination with a collecting device and analysis device, the steps from bacteria collection to gene extraction can be automated in two kinds of small chip. Since no manual operation is involved in the bacteria spore treatment or the gene extraction step, any person can safely conduct the analysis. Further, since the interior of the compact chip is free from mechanical parts such as valves, a chip can be provided which is suitable for single-use applications. Furthermore, as a result of miniaturization of the volume of the reaction chamber and the channels by microfabrication, it is possible to obtain such advantages as reduction in the amount of reagents and in cost as well as rapid temperature control, rapid mixing, and homogeneous reaction. Moreover, since the reagent for just one detection is pre-embedded in a disposable analysis chip and the analysis chip is provided to the user in a refrigerated or frozen state, extremely simple and fast gene detection can be attained. Further, such as chip can be disposed of with the reagents after the analysis is completed.

It should be further understood by those skilled in the art that although the foregoing description has been made on embodiments of the invention, the invention is not limited thereto and various changes and modifications may be made without departing from the spirit of the invention and the scope of the appended claims.

While the above examples were described using one reaction chamber 390 in the analysis chip 300, depending on the subject to be analyzed and the like, the number of reaction chambers 390 may be increased. In such case, primers corresponding to each bacteria of the object to be detected are necessary, which requires multiple gene-amplification reagent A storage chambers containing the primers. In addition, while it is necessary to switch the irradiation position of the excitation light from the light source 450 with respect to the reaction chamber 390 in order to detect the reactions in the multiple reaction chambers 390, such a configuration has the advantage of allowing a plurality of bacteria to be simultaneously detected on a single analysis chip.

In the above examples, an example was illustrated in which there is one chip setting section where the collecting chip 200 and the analysis chip 300 are placed. However, to simultaneously conduct the collecting chip 200 treatment and the analysis chip 300 treatment in parallel, two chip setting sections may be provided. In the collecting chip 200 treatment step, the optical detection system is not necessary, so that two chip setting sections can be provided by integrating the fluid system and temperature control system. Although the size of the device does slightly increase, the treatment time for multiple detection objects can be shortened by simultaneously treating with the collecting chip and analysis chip.

In the above examples, a piezoelectric element such as a quartz oscillator or a surface acoustic wave element may be provided at the bottom of the analysis chip 300. Since the piezoelectric element changes the weight applied on the electrode to changes in oscillating frequency in a quantitative manner, minute changes in weight under a reaction atmosphere can be measured on a continuous basis. Thus, various nucleotides having known base sequences are fixed on the piezoelectric element. The method of fixing is preferably as follows.

A glass thin film is formed on the electrode of the piezoelectric element by sputtering, vapor deposition, or the like. The glass preferably has a main ingredient of SiO₂ that is most adhesive to electrode elements such as chromium or titanium. By applying aminopropyltrimethoxy silane (APS) to this glass thin film and baking at about 120 to 160° C., amino groups are fixed on the surface of the glass thin film. The thickness of the electrode and the glass thin film is preferably 0.1 to 1 μm, respectively. This is because if the thickness of the two exceeds 1 μm, the frequency response of the piezoelectric element becomes less responsive. Furthermore, nucleotide is plated on the glass thin film of which amino groups have been coated, and incubated at 37° C. and a humidity of 90% for 1 hour in an incubator. Then, by irradiating UV of 60 mJ/cm² using a UV crosslinker, nucleotide is strongly fixed to the piezoelectric element.

The steps as far as the extraction of a gene from the sample are the same as in the above examples. When the temperature of the gene transported to the reaction chamber 390 is increased to about 94° C. by the temperature control mechanism 415, the gene becomes heat-denatured and forms a single strand. When this single strand gene is bound to the nucleotide fixed on the bottom of the chip, the oscillation frequency of the piezoelectric element changes. Thus, by measuring this change in frequency, the sequence of the gene complementary to the fixed nucleotide can be read.

When a piezoelectric element is used in a solution, a change in the solution temperature of 1° C. leads to a change in frequency of 15 to 30 Hz, and thus precise control of the solution temperature is essential. In this example, however, neither the gene-amplification reagent nor the temperature cycle is needed, and thus it has an advantage that time required for detection becomes shorter.

### ADVANTAGES OF THE INVENTION

According to the present invention, an analysis system is provided which can simply conduct from microorganism collection to gene detection on the chip safely and in a short time.

1. A bio-analysis system which collects microorganisms floating in the air by suction with a collecting device to analyze a gene contained in the microorganisms, comprising:
a collecting chip which is placed in the collecting device to collect the microorganisms on a collecting material and is then removed from the collecting device and placed in an analysis device;
an analysis device which transports a reagent in the collecting chip with the collecting chip placed in the analysis device to conduct in the collecting chip germination of spores in the microorganisms and a cell membrane lysis treatment; and
an analysis chip which comprises a sample reservoir, a gene extraction area filled with a gene binding carrier, a waste solution chamber filled with absorbent, a washing solution storage chamber which stores a washing solution, an eluting solution storage chamber which stores a gene eluting solution, a gene-amplification reagent storage chamber which stores a gene-amplification reagent, and a reaction chamber which amplifies and detects the gene, each of which is formed by a channel, and a sample injection port open to a front face and a chip port open to a rear face, wherein
a part of the solution treated by the collecting chip is moved to the analysis chip, and the treatments from gene extraction to detection are conducted on the analysis chip by liquid transport means of the analysis device.

2. The bio-analysis system according to claim 1, wherein an interior side of the waste solution chamber is subjected to a hydrophilic treatment.

3. The bio-analysis system according to claim 1, wherein a cross-sectional area of the channels other than the gene-amplification reagent storage chamber is smaller than the cross-sectional area of the gene-amplification reagent storage chamber.

4. The bio-analysis system according to claim 1, wherein a cross-sectional area of the channels other than the gene-amplification reagent storage chamber is no greater than ¼ of the cross-sectional area of the gene-amplification reagent storage chamber.

5. The bio-analysis system according to claim 1, wherein a channel of the gene-amplification reagent storage chamber is U-shaped.

6. The bio-analysis system according to claim 1, wherein the gene-amplification reagent storage chamber is located at an upstream position of the sample reservoir and the washing solution storage chamber.

7. The bio-analysis system according to claim 1, wherein a gas of inert nitrogen, helium or argon is injected into the analysis chip to conduct transportation.

8. The bio-analysis system according to claim 1, wherein two kinds of absorbent are filled into the waste solution chamber.

9. The bio-analysis system according to claim 1, wherein an interior side of the waste solution chamber has a resin surface modified by irradiation of plasma.
